(19) United States
(12) Patent Application Publication
(10) Pub. No.: US 2013/0140208 A1
(43) Pub. Date: Jun. 6, 2013

(54) MEDICAMENT CARTRIDGES WITH NON-STANDARD DIMENSIONS

(71) Applicant: Kerstine Hemmann, Frankfurt (DE)

(72) Inventor: Kerstine Hemmann, Frankfurt (DE)

(73) Assignee: SANOFI-AVENTIS DEUTSCHLAND GMBH, Frankfurt am Main (DE)

(21) Appl. No.: 13/810,502

(22) PCT Filed: Jul. 18, 2011
(86) PCT No.: PCT/EP11/62277

§ 371 Date: Feb. 19, 2013

(30) Foreign Application Priority Data
Jul. 19, 2010 (US) 61365457
Sep. 2, 2010 (EP) 10174997.6

(51) Int. Cl. A61J 1/00; B65B 3/00

(52) U.S. Cl. CPC A61J 1/00; B65B 3/003
USPC 206/438; 53/476

(57) ABSTRACT

A method of closing the distal end of an ampoule is shown. The ampoule has a variable diameter head portion having a locating surface and an opening. A septum is positioned on the opening and secured to the head portion with a ferrule using a plunger. The plunger exerts a force in a proximal direction to press the ferrule on the head portion until the press causes the ferrule to contact the locating surface. Further, the ampoule for use in a cartridge for a drug delivery device has non-standard dimensions to provide a coding system to reduce the risk of a user dispensing the wrong medicament from the drug delivery device.

### CROSS REFERENCE TO RELATED APPLICATIONS

The present application is a U.S. National Phase Application pursuant to 35 U.S.C. §371 of International Application No. PCT/EP2011/062277 filed Jul. 18, 2011, which claims priority to U.S. Provisional Patent Application No. 61/365,457 filed Jul. 19, 2010 and European Patent Application No. 10174997.6 filed Sep. 2, 2010. The entire disclosure contents of these applications are herewith incorporated by reference into the present application.

### FIELD OF INVENTION

The present disclosure is generally directed to drug delivery devices and reservoirs (i.e., ampoules and cartridges), particularly reservoirs containing a medicament. More particularly, the present application is generally directed to cartridges having non-standard dimensions that can provide a coding system for drug delivery device components to prevent unwanted cross use. As just one example, such medicament cartridges may comprise an ampoule having a distal head portion having multiple outside diameters, preferably having at least two different diameters. The disclosure also includes an improved method of manufacturing a finished cartridge using the improved ampoule. Exemplary medical delivery devices that accept the cartridges of this disclosure include, but are not limited to, syringes, pen type injection syringes, pumps, inhalers, or other similar injection or infusing devices that require at least one reservoir containing at least one medicament.

### BACKGROUND

Medicament reservoirs such as ampoules, cartridges, or vials are generally known. Such reservoirs are especially used for medicaments that may be self administered by a patient. For example, with respect to insulin, a patient suffering from diabetes may require a certain amount of insulin to either be injected via a pen type injection syringe or infused via a pump. With respect to certain known reusable pen type drug delivery devices, a patient loads a cartridge containing the insulin into a proximal end of a cartridge holder. After the cartridge has been correctly loaded, the user may then be called upon to select a dose of medicament. Multiple doses may be dosed from the cartridge. Where the drug delivery device comprises a reusable device, once the cartridge is empty, the cartridge holder is disconnected from the drug delivery device and the empty cartridge is removed and replaced with a new cartridge. Most suppliers of such cartridges recommend that the user dispose of the empty cartridges properly. Where the drug delivery device comprises a disposable device, once the cartridge is empty, the user is recommended to dispose of the entire device.

Such known self-administration systems requiring the removal and reloading of empty cartridges have certain limitations. For example, in certain generally known systems, a user simply loads a new cartridge into the delivery system without the drug delivery device or without the cartridge having a mechanism of preventing cross use of an incorrect cartridge. That is, the drug delivery device does not have a mechanism for determining if the medicament contained in the cartridge is indeed the correct type of medicament to be administered by the patient. Alternatively, certain known drug delivery devices do not present a mechanism for determining if the correct type of medicament within the cartridge should be used with that particular drug delivery system. This potential problem could be exacerbated given that certain elderly patients, such as those suffering from diabetes, may have limited manual dexterity. Identifying an incorrect medicament is quite important, since the administration of a potentially incorrect dose of a medicament such as a short acting insulin in lieu of a long insulin could result in injury or even death.

Some drug delivery devices or systems may use a color coding scheme to assist a user or care giver in selecting the correct cartridge to be used with a drug delivery device. However, such color coding schemes pose challenges to certain users, especially those users suffering from poor eyesight or color blindness: a situation that can be quite prevalent in patients suffering from diabetes.

Another concern that may arise with such disposable cartridges is that these cartridges are manufactured in essentially standard sizes and manufactured to comply with certain recognized local and international standards, for example ISO Standard 11608-3 2001. Consequently, such cartridges are typically supplied in standard sized cartridges (e.g., 3 ml cartridges). Therefore, there may be a variety of cartridges supplied by a number of different suppliers and containing a different medicament but they may fit a single drug delivery device. As just one example, a first cartridge containing a first medicament from a first supplier may fit a medical delivery device provided by a second supplier. As such, a user might be able to load and then dispense an incorrect medicament (such as a rapid or basal type of insulin) into a drug delivery device without being aware that the medical delivery device was perhaps neither designed nor intended to be used with such a cartridge.

As such, there is a growing desire from users, health care providers, care givers, regulatory entities, and medical device suppliers to reduce the potential risk of a user loading an incorrect drug type into a drug delivery device. There is also, therefore, a desire to reduce the risk of dispensing an incorrect medicament (or the wrong concentration of the medicament) from such a drug delivery device.

There is, therefore, a general need to physically dedicate or mechanically code a cartridge to its drug type and design an injection device that accepts or works with the dedication or coded features provided on or with the cartridge so as to prevent unwanted cartridge cross use. Similarly, there is also a general need for a dedicated cartridge that allows the medical delivery device to be used with an authorized cartridge containing a specific medicament while also preventing undesired cartridge cross use.

There is also a general need to provide a dedicated cartridge that is difficult to tamper with so that the cartridge may not be compromised in that the cartridge can be used with an unauthorized drug or drug delivery device. Because such cartridges may be difficult to tamper with, they may also reduce the risk of counterfeiting: i.e., making it more difficult for counterfeiters to provide unregulated counterfeit medicament carrying products.

It is an aim of the invention to reduce the risk of a user dispensing the wrong medicament from the drug delivery device.

### SUMMARY

The aim is achieved by an ampoule for use in a cartridge containing a medicament for use in a drug delivery device, the ampoule having non-standard dimensions to provide a coding system to reduce the risk of a user dispensing the wrong medicament from the drug delivery device. The cartridge contains an ampoule having a head portion with variable diameters.

One embodiment of the ampoule for containing a medicament comprises a constant diameter body, a neck portion and a head portion located distally of the neck portion and having a non-constant, variable diameter. In other words, the head portion is not a tube having a constant diameter. Rather the head portion has a section having a diameter which differs from the diameter of another section of the head portion. The non-constant or variable diameter serves as coding feature. The ampoule may have a proximal and distal end, where the distal end of the ampoule has the head portion that defines an opening where a medicament, such as insulin, can be dispensed through a needle cannula, for example. The transition between the head portion and the neck portion may be an inwardly converging shoulder. The neck portion may enlarge into a constant diameter reservoir or body that comprises the major portion of the ampoule volume. The ampoule may be a part of a cartridge, which in addition to the ampoule, may also contains a septum mounted across the distal opening of the ampoule by a ferrule and a piston that is slidably positioned inside the proximal end of the ampoule and forms a seal to contain the medicament within the ampoule.

One embodiment of the ampoule for containing a medicament comprises a constant diameter body having a proximal end and a distal end; a neck portion having a diameter DND at the distal end; and a head portion located distally of the neck portion and having a variable, non-constant diameter.

In one embodiment the head portion has a first diameter and a second diameter, where the first diameter is less than the second diameter. The diameter DND of the neck portion may be less than the first and second diameters.

According to exemplary arrangements, both a starting ampoule and a finished cartridge are provided having “non-standard” dimensions (i.e., non-ISO standard), where the finished cartridge is intended for use with a matched reservoir holder of a drug delivery device. A system comprised of such non-standard ampoules and finished cartridges allows for a coding system that distinguishes cartridges containing the same medicament at different concentration levels and/or cartridges containing different medicaments. A matching cartridge holder accepts only the non-standard finished cartridge.

A standard cartridge is comprised of ampoule having a proximal and distal end, where the distal end of the ampoule has a head portion that defines an opening where a medicament, such as insulin, can be dispensed through a needle cannula. The head portion has a pierceable septum fixed to the ampoule by a ferrule that is typically crimp fitted to the head portion. Importantly, it is noted that the head portion of a standard ISO ampoule is “bottle shaped” and has a constant outside diameter that terminates in a neck portion before enlarging into a constant diameter reservoir that comprises the major portion of the ampoule volume. The finished cartridge, in addition to the ampoule, also contains a bung, stopper, or piston that is slidably position inside the proximal end of the ampoule and forms a seal to contain the medicament within the ampoule. Typically, the ampoule is formed from glass, but will work with any known materials of construction, such as, plastics or like materials.

A “standard cartridge” is known to those skilled in the art of drug delivery devices to be one that comports with the dimensions set by International Standard ISO 11608-3:2000A. For a cartridge nominally holding 3 ml, the ISO standard specifies the following standard dimensions:

| | |
|---|---|
| L (total length) | 63.90 + 0.30 |
| NL (length of distal neck) | 6.30 max. |
| D (distal diameter) | 8.0 max. |

The 8 mm max. dimension shown above for the distal diameter of the cartridge is measured as a cross-section of the head portion of the distal end of ampoule and includes the thickness of the ferrule. Thus, this dimension D is a function of the wall thickness of the ampoule, the thickness of ferrule, and the size of the distal opening of the ampoule. As mentioned, this dimension D is a uniform diameter (non-variable) that begins at the very distal end of the ampoule and continues until termination at the neck portion. FIG. 2 shows a cross-sectional view of a “standard” ampoule as part of a finished ISO standard cartridge. Although the ISO standards provide dimensions and shapes of the starting ampoules, manufacturing tolerances of +0.20 mm are industry normal. In order to use such a standard cartridge to administer medicament by injection, the drug delivery device typically has a cartridge holder with an internal diameter of 8.2 mm or greater to ensure the standard cartridge will fit into the cartridge holder portion of the injection device. Like the ampoule, the distal end of a standard cartridge holder has single, constant diameter cavity designed to accept the constant diameter distal head portion of the finished cartridge.

In one aspect, a medicament ampoule is provided comprising a constant diameter body having a proximal end and a distal end. There is also a neck portion having a diameter DND at the distal end of the ampoule, which defines a transition point between the constant diameter body and the distal end of the ampoule. At the very distal end of the ampoule is a head portion located distally of the neck portion. This head portion is unique in that is has a variable, non-constant diameter. The ampoule also has a total length L and an outside diameter OD that defines the constant diameter body portion of the ampoule. Preferably, the head portion comprises at least two different diameters that are separate from the diameter of the neck portion DND. This variable diameter head portion is distinguishable from the single or uniform head diameter found on an ISO standard ampoule. Preferably, the head portion of my ampoule has a first and a second head diameter, where the first head diameter is smaller than the second head diameter, and both the first and second diameters are larger than the neck diameter DND.

In another aspect, a finished medicament cartridge is provided comprising an ampoule having the characteristics described above with the addition of a ferrule that partially encloses a septum. The ferrule is fixed to the head portion at the distal end of the ampoule preferably by crimping the ferrule around both the first and second head portion diameters. A medicament is sealed inside the ampoule by a piston that is slideably positioned within the proximal portion of the ampoule. Most preferably, the ferrule conforms exactly to the dimensions of the head portion of the ampoule and includes both diameters, but not the neck portion. The at least two outside distal diameters, D1 and D2, of the distal end of the finished cartridge are measured in same manner as defined by the ISO standards (i.e., the cross-section of the distal end of the ampoule including the ferrule). Preferably, D1 is in the range from about 7.50 to about 8.00 mm, and D2 is in the range from about 5.7 to about 6.5 mm. Although either D1 or D2 could equal that specified in the ISO Standards, the ampoule and/or cartridge would still be considered “non-standard” because the ampoule was manufactured with a variable diameter head portion having at least two distinct diameters in the head portion, exclusive of the neck diameter. Likewise, even though the total length L of such an ampoule and/or cartridge could be equal to the ISO standard set forth above, the cartridge would still be considered “non-standard” because the head portion would have at least two different diameters. The ISO standard, contrary to the disclosure, specifies only a single uniform diameter for the head portion of the finished cartridge.

Manufacturing or simply providing a drug delivery device, either disposable or refillable, that has a cartridge holder with an internal distal cavity that matches the contour (i.e., the variable head portion diameters) of the finished cartridges of this disclosure (i.e., “non-standard”), but will not accept a “standard” 3 ml cartridge as defined by ISO, will provide a needed coding feature. This exclusion of standard cartridges provides a way to prevent or reduce the potential risk of a user loading an incorrect drug type into a drug delivery device. Likewise, this prevents undesired cartridge cross use.

The disclosure also concerns a system of medicament cartridges. The system may comprise a first cartridge which comprises a first ampoule and contains a first medicament; and a second cartridge comprising a second ampoule and containing a second medicament. The second diameter of the first and second ampoules is the same dimension and the first diameter of the first and second ampoules is a different dimension. In one embodiment the first medicament has a first concentration and the second medicament has a second concentration, where the second concentration is not equal to the first concentration. In an alternative embodiment the first medicament in the first cartridge is different than the second medicament in the second cartridge

One embodiment of a system of medicament cartridges comprises at least two cartridges. A first cartridge contains a first concentration of medicament. A second cartridge contains a second concentration of medicament. The first and second cartridges each comprise an ampoule having a constant diameter body having a proximal end and a distal end, a neck portion having a diameter DND at the distal end, and a head portion located distally of the neck portion and having at least two diameters, D1 and D2; where D2 of each ampoule of each cartridge is the same dimension and D1 of each ampoule of each cartridge is a different dimension. The second concentration may be not equal to the first concentration. The medicament in the first cartridge may be different than the medicament in the second cartridge.

The first and second cartridges may further comprises a septum mounted across the distal opening of the ampoule by a ferrule which conforms to the dimensions of the head portion. The ferrule may include the first and second diameters of the respective ampoule.

Accordingly, the disclosure includes a system of cartridges, defined as two or more cartridges, where a first cartridge can contain a first concentration of medicament and a second cartridge can contain a second concentration of medicament. The first and second cartridges in the system each comprise an ampoule having a proximal portion, a total length L, and a head portion having at least two measurable diameters, exclusive of the neck diameter. A ferrule partially enclosing a septum is fixed to the ampoule and preferably surrounds both head diameters. The distal head diameters can be varied to distinguish the different concentrations and/or different medicaments contained in the cartridges. Preferably, the second concentration of medicament is not equal to the first concentration. Alternatively, the medicament in the first cartridge could be different than the medicament in the second cartridge. Of course, the system could include a number of cartridges, where each cartridge has the same D1, but a different D2. In such a system, different medicament can be coded or matched to different D2 diameters. Likewise, the system could include a number of cartridges, where each cartridge has the same D2, but a different D1. In such a system, different medicament can be coded or matched to different D1 diameters. Improper cartridge use can be avoided by providing drug delivery devices with cartridge holders that only accept one or more cartridges with the matching head portion diameters. This is accomplished by varying the internal dimensions and/or design at the distal end of the cartridge holders.

The disclosure also relates to an improved method of assembling the finished cartridge. A method of closing the distal end of an ampoule may comprise providing an ampoule having a variable diameter head portion having a locating surface and an opening; positioning a septum on the opening; and securing the septum to the head portion with a ferrule using a plunger, where the plunger exerts a force in a proximal direction to press the ferrule on the head portion until the press causes the ferrule to contact the locating surface. A rolling plate may exert a force in a distal direction to crimp the ferrule to the head portion.

In the production process for closing the distal end of a conventional ampoule there is no locating surface because the head portion is cylindrical in shape and is uniform in cross-section dimension until it tapers into the neck portion. As such, the sealing of the ferrule to head portion and fixation of the septum is exclusively controlled by force, which must be large enough in order to sufficiently compress the septum in order to thus ensure leak-tightness, but it cannot be so large that the overhang of the ferrule material (typically aluminum foil) has a larger surface than the opposite surface that is available on the glass body. This would lead to an edge of the ferrule that is unclean, “frayed” or sticks out. In the situations where there are changes in the assembly process, for example, where the material of the septum or ferrule is changed because of alternative suppliers or where there is a wear of the assembly tools, the required force must be determined anew by an iterative trial and error process that leads to production waste.

The method makes use of a locating surface formed by a horizontal or substantially horizontal surface associated with either the first or second diameters in the head portion of the cartridge. The machine used to seal the ferrule to the ampoule employs a plunger tool that is forced downwardly around the head portion of the ampoule until the surface of the aluminum cap reaches the locating surface of the diameter defined by either D1 or D2 on the head portion of the ampoule. In this manner the production or assembly process using a locating surface is dependent on the position of the locating surface and on the wear and tear of the tool, which simplifies the production process.

The drug delivery device and the non-standard cartridges described herein can be considered a drug delivery system. Such a drug delivery system can be reusable, meaning the cartridge can be replaced when empty, or the system can be non-reusable (disposable), meaning the cartridge cannot be replaced and the entire system is thrown away when the cartridge is empty.

In any of the arrangements described above, it is possible to add mechanical coding features to the non-standard cartridges, for example, coded labels or ring/bands/collars attached to the proximal end of the ampoule. These as well as other advantages of various aspects will become apparent to those of ordinary skill in the art by reading the following detailed description, with appropriate reference to the accompanying drawings. The scope of the invention is defined by the content of the claims. The invention is not limited to specific embodiments but comprises any combination of elements of different embodiments. Moreover, the invention comprises any combination of claims and any combination of features disclosed by the claims.

### BRIEF DESCRIPTION OF THE DRAWINGS

Exemplary embodiments are described herein with reference to the drawings, in which:

FIG. 1 illustrates an exemplary pen type drug delivery device;

FIG. 2 illustrates a cross-sectional view of an ISO standard drug cartridge;

FIG. 3 is a cross-sectional view of exemplary drug cartridge in accordance with our proposed concept;

FIG. 4 is a cross-sectional view of another exemplary drug cartridge in accordance with our proposed concept;

FIG. 5 is a cross-sectional view of the manufacturing process for an ISO standard cartridge; and

FIG. 6 is a cross-sectional view of the manufacturing process for exemplary drug cartridge in accordance with our proposed concept.

### DETAILED DESCRIPTION

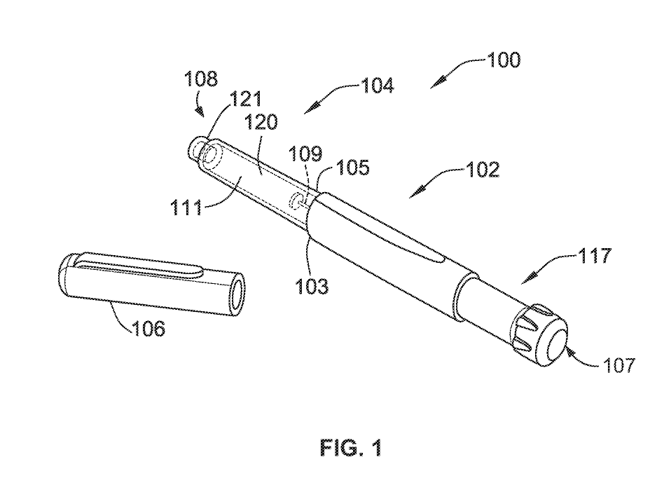

FIG. 1 illustrates a drug delivery device 100 in the form of a pen type syringe. This drug delivery device 100 comprises a dose setting mechanism 102, a cartridge holder 104, and a removable cap 106. A proximal end 105 of the cartridge holder 104 and a distal end 103 of the dose setting mechanism 102 are removably secured together. The pen type syringe may comprise a re-usable or a disposable pen type syringe. Where the syringe comprises a reusable device, the cartridge holder 104 and the dose setting mechanism 102 are removably coupled together. In a disposable device, they may be permanently coupled together. In FIG. 1, the dose setting mechanism 102 comprises a spindle 109, such as a threaded spindle that rotates when a dose is injected.

To inject a previously set dose, a double ended needle assembly (not shown) is attached to a distal end 108 of the cartridge holder 104. Preferably, the distal end 108 of the cartridge holder 104 comprises a thread 121 (or other suitable connecting mechanism such as a snap lock, snap fit, form fit, or bayonet lock mechanism) so that the needle assembly may be removably attached to the distal end 108 of the cartridge holder 104. When the drug delivery device 100 is not in use, the removable cap 106 can be releasably retained over the cartridge holder 104.

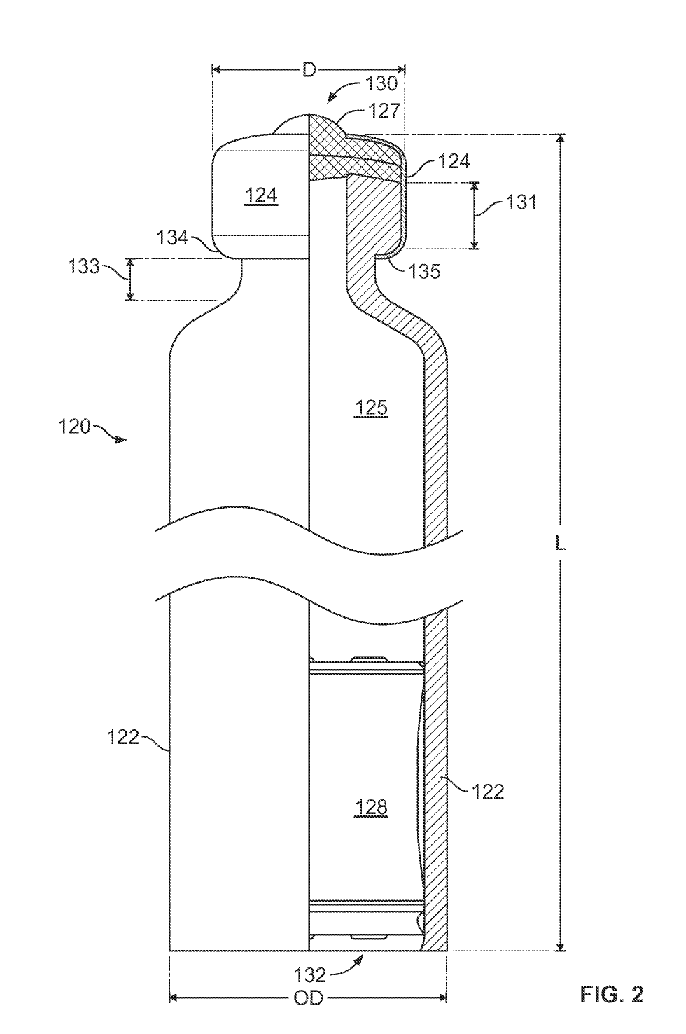

An inner cartridge cavity 111 defined by the cartridge holder 104 is dimensioned and configured to securely receive and retain a non-standard cartridge 120. FIG. 2 illustrates a partial cross-sectional view of the distal end of a standard ISO cartridge 120 having a uniform, non-variable, diameter D for the head portion 131 that may be used with the drug delivery device 100 illustrated in FIG. 1 provided the inner cavity 111 is contoured and/or conformed to matched the uniform shape of the head portion 131 of the standard ISO cartridge 120. The cartridge 120 includes an ampoule 122 extending from a distal end 130 to a proximal end 132.

The distal end 130 is defined by the combination of the head portion 131 and a neck portion 133, where the transition is an inwardly converging shoulder 135.

At the distal end 130, the ampoule 122 includes a constant and uniform diameter head portion 131 having diameter D located distally of the neck portion 133. An annular bead 134 extends circumferentially thereabout at the extreme distal end of shoulder 135. A pierceable seal or septum 127 is securely mounted across the open distal end of the ampoule 122. The septum 127 may be held in place by a metallic sleeve or ferrule 124. This ferrule 124 may be crimped around the circumferential bead 134 at the distal end of the neck portion 133. The medicament 125 is pre-filled into the cartridge 120 and is retained within the cartridge 120, in part, by the pierceable seal or septum 127, the ferrule 124, and a piston 128. The piston 128 is in sliding fluid-tight engagement with the inner tubular wall of the ampoule 122. Axially directed forces acting upon the piston 128 during dose injection or dose administration urges the medication 125 from the cartridge 120 though a double ended needle (not shown) mounted onto the distal end 108 of the cartridge holder 104 and into the injection site. Such axial forces may be provided by the spindle 109.

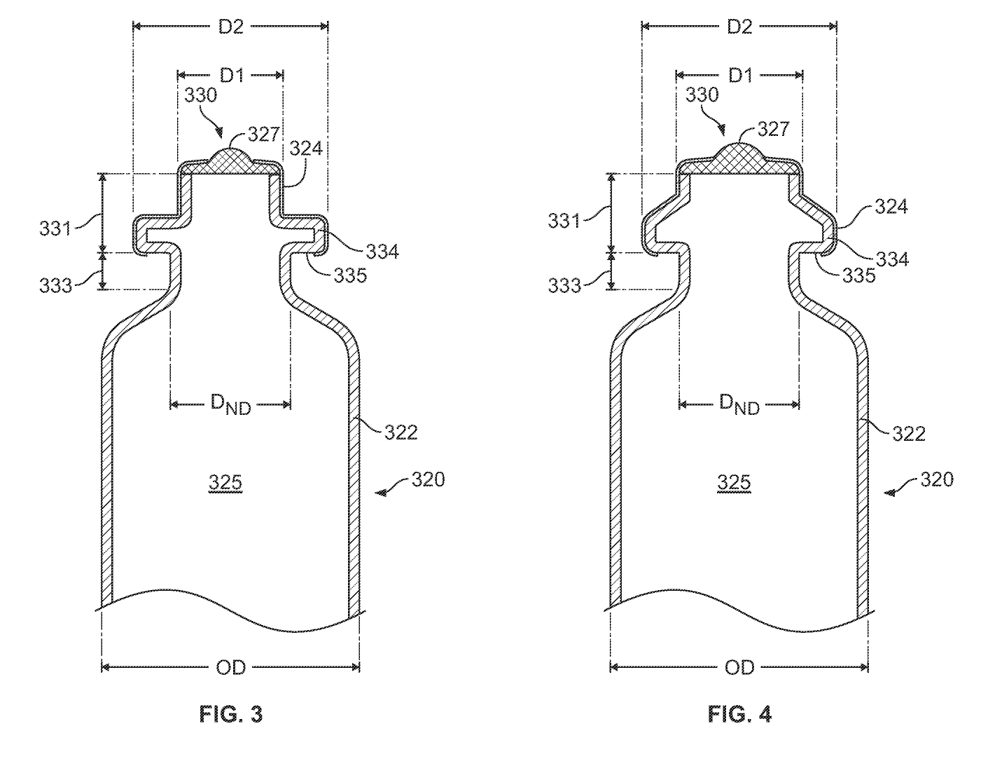

Turning to FIGS. 3 and 4, which show examples of the ampoule 322 and finished cartridges 320 of this disclosure, each is characterized in that the head portion 331 has a variable diameter separate and apart from the diameter DNP of neck portion 333. At the distal end 330, the ampoule 322 includes a non-uniform head portion 331 having, at a minimum at least two different diameters, shown as D1 and D2 located distally of the neck portion 333. In each of the embodiments shown in FIGS. 3 and 4 an outside distal diameter D1 is smaller than D2. As with the standard ISO cartridge, the cartridges of the disclosure, as depicted in these figures, has an annular bead 334 extending circumferentially at the extreme distal end of shoulder 335, a pierceable seal or septum 327 securely mounted across the open distal end of the ampoule 322 by a metallic sleeve or ferrule 324, which may be crimped around the circumferential bead 334 at the distal end of the neck portion 333. The ampoule 322 contains a medicament 325 pre-filled into the cartridge 320 that is retained within the cartridge 320, in part, by the pierceable seal or septum 327, the ferrule 324, and a slidable piston (not shown), but which can be the same as piston 128 shown in FIG. 2.

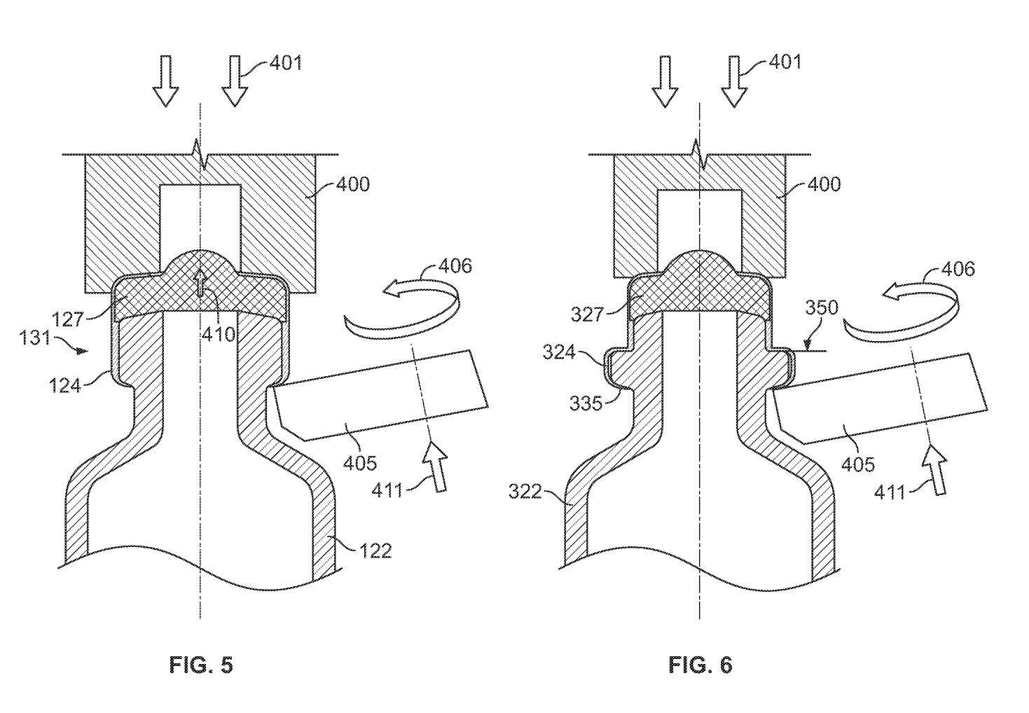

The disclosure also covers an improved manufacturing or filling process that is possible as a result of the variable diameter head portion 331 of ampoule 322. The improved manufacturing process makes use of a locating surface 350 located on the head portion 331, preferably on a horizontal or nearly horizontal surface, such as locating surface 350 shown in FIG. 6. In contrast, FIG. 5 illustrates a manufacturing process for an ISO standard cartridge 120 where there is no locating surface because of the uniform constant diameter of the head portion 131 of the ampoule 122. In the manufacturing process a press 400 exerts a force in direction 401 to secure the ferrule 124 to head portion 131. The plunger is spring-loaded and is driven down until the counter pressure of septum 127 that is compressed by this process reaches a force 410. Rolling plate 405 exerts an upward force 411 and moves in direction 406 to attach the overhang of ferrule 124 that is created by the compression of the septum 127 to the head portion 131 of ampoule 122. In the improved process, as shown in FIG. 6, the press 400 is forced downward until the press 400 causes the ferrule 324 to encounter the locating surface 350 of ampoule 322. The same rolling plate 405 is used to secure ferrule 324 to the shoulder 335. This improved production process is only dependent on the position of the locating surface 350 and the wear and tear of the tool, but not the counter pressure exerted by the septum 327, which simplifies the production process.

A portion of the cartridge holder 104 defining the cartridge holder cavity 111 is of substantially uniform diameter. This inner diameter is preferably slightly greater than the outer diameter OD at the proximal end of the main body of cartridge 320. The distal interior of the cartridge holder 104 is configured, molded, formed or otherwise designed to conform to the variable diameter head portion 331 of the cartridges of the disclosure. In this manner, when the cartridge 320 is loaded into the cavity 111 of the cartridge holder 104 and the cartridge holder 104 is then connected to the dose setting member 102, the cartridge 320 will be securely held within the cartridge cavity 111. More particularly, because the distal interior of the cartridge holder 104 is designed to match the variable diameter of the neck portion 331 of the cartridge 320, only matching cartridges 320 and cartridge holders 104 will allow compatible fit and attachment to the dose setting mechanism 102 of the drug delivery device 100.

A number of doses of a medicament 325 may be dispensed from the cartridge 320. It will be understood that the cartridge 320 may contain a type of medicament 325 that must be administered often, such as one or more times a day. One such medicament 325 is insulin. The dose setting mechanism 102 comprises a dose setter 117 at the proximal end 107 of the drug delivery device 100. In one preferred arrangement, the dose setter 117 may extend along the entire length of the dose setting mechanism. The dose setter 117 may be rotated by a user to set a dose.

To administer a dose that may be set by rotating the dose setter 117, the user attaches the needle assembly comprising a double ended needle on the distal end 108 of the cartridge holder 104. In this manner, the needle assembly pierces the seal 127 of the cartridge 120 and is therefore in liquid communication with the medicament 125. The user pushes on the dose setter 117 to inject the set dose. The same dose setting and dose administration procedure is followed until the medicament 125 in the cartridge is expended and then a new cartridge 120 must be loaded in the device. To exchange an empty cartridge 120, the user is called upon to remove the cartridge holder 104 from the dose setting mechanism 102.

A coding system comprising the non-standard cartridges 320 of the disclosure for use with a drug delivery system, such as drug delivery device 100, is provided. In an example, a system of cartridges 320 are manufactured where the head portion 331 of the cartridges 320 are variable in diameter having at least two measurable diameters D1 and D2, this being contrary to the constant and uniform distal diameter D of a “standard cartridge” 120 as required by the ISO standards. As such, each cartridge 320 could have the same D1, but a different D2, or vice versa, with either or both of D1 or D2 coded or matched to a different medicament 325 or concentration of medicament 325. Because the cartridge holder 104 is manufactured to fit the non-standard distal diameters D1, D2 for each cartridge system, an attempt to insert or use a standard cartridge 120 will fail and as such it will not be possible to accidentally use a standard cartridge 120 in place of the non-standard cartridge 320.

Although aimed primarily at the insulin market, the proposed non-standard cartridge schemes may apply to other drugs. Likewise, the coding system may apply to various drug delivery devices 100.

The proposed cartridge system results in a number of advantages. For example, the proposed system help to assist a user to ensure that a given drug delivery device component is only attached to a drug delivery device component for which it is intended. The system also results in a low cost coding mechanism since the manufacture of cartridges 320 with varying distal diameters D1, D2 and matching holders 104 does not require a large number of parts and can be manufactured in a cost effective manner. Moreover, there are quite a large number of different possible dimensions for the variable diameter head portion 331 that can be used. Consequently, with the proposed non-standard cartridge schemes, a large number of medicaments 325 can be distinguished from one another.

In given embodiments, the coding may be designed to block all incorrect reservoirs from being inserted into an incorrect cartridge holder 104. In alternative embodiments, the coding may be designed to block reservoirs of a given type, but not all types of reservoirs. For example, in an embodiment, the coding may block only reservoirs not intended for the housing and that comprise dangerous drugs. For instance, a short-acting drug could be fitted into a device intended for long-acting drugs, but not vice versa. As another example, a low concentration drug could be fitted into a device intended for high concentration drugs, but not vice versa.

Exemplary embodiments have been described. However, as those of skill in the art will recognize certain changes or modifications to such arrangements may be made. Those skilled in the art will understand, however, that further changes, modifications, revisions and/or additions may be made to the presently disclosed arrangements without departing from the true scope and spirit of the present disclosure, which is defined by the claims.

The term “drug” or “medicament”, as used herein, means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, a antibody, an enzyme, an antibody, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyhepta [symbol] decanoyl) human insulin.

Exendin-4 for example means Exendin-4(1-39), a peptide of the sequence H His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:
H-(Lys)4-des Pro36, des Pro37 Exendin-4(1-39)-NH2,
H-(Lys)5-des Pro36, des Pro37 Exendin-4(1-39)-NH2,
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(O2)25, Asp28] Exendin-4(1-39),
des Pro36 [Met(O)14 Trp(O2)25, IsoAsp28] Exendin-4(1-39); or
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(O2)25, Asp28] Exendin-4(1-39),
des Pro36 [Met(O)14 Trp(O2)25, IsoAsp28] Exendin-4(1-39),
wherein the group -Lys6-NH2 may be bound to the C-terminus of the Exendin-4 derivative;
or an Exendin-4 derivative of the sequence
H-(Lys)6-des Pro36 [Asp28] Exendin-4(1-39)-Lys6-NH2,
des Asp28 Pro36, Pro37, Pro38Exendin-4(1-39)-NH2,
H-(Lys)6-des Pro36, Pro38 [Asp28] Exendin-4(1-39)-NH2,
H-Asn-(Glu)5des Pro36, Pro37, Pro38 [Asp28] Exendin-4(1-39)-NH2,
des Pro36, Pro37, Pro38 [Asp28] Exendin-4(1-39)-(Lys)6-NH2,
H-(Lys)6-des Pro36, Pro37, Pro38 [Asp28] Exendin-4(1-39)-(Lys)6-NH2,
H-Asn-(Glu)5-des Pro36, Pro37, Pro38 [Asp28] Exendin-4(1-39)-(Lys)6-NH2,
H-(Lys)6-des Pro36 [Trp(O2)25, Asp28] Exendin-4(1-39)-Lys6-NH2,
H-des Asp28 Pro36, Pro37, Pro38 [Trp(O2)25] Exendin-4(1-39)-NH2,
H-(Lys)6-des Pro36, Pro37, Pro38 [Trp(O2)25, Asp28] Exendin-4(1-39)-NH2,
H-Asn-(Glu)5-des Pro36, Pro37, Pro38 [Trp(O2)25, Asp28] Exendin-4(1-39)-NH2,
des Pro36, Pro37, Pro38 [Trp(O2)25, Asp28] Exendin-4(1-39)-(Lys)6-NH2,
H-(Lys)6-des Pro36, Pro37, Pro38 [Trp(O2)25, Asp28] Exendin-4(1-39)-(Lys)6-NH2,
H-Asn-(Glu)5-des Pro36, Pro37, Pro38 [Trp(O2)25, Asp28] Exendin-4(1-39)-(Lys)6-NH2,
H-(Lys)6-des Pro36 [Met(O)14, Asp28] Exendin-4(1-39)-Lys6-NH2,
des Met(O)14 Asp28 Pro36, Pro37, Pro38 Exendin-4(1-39)-NH2,
H-(Lys)6-desPro36, Pro37, Pro38 [Met(O)14, Asp28] Exendin-4(1-39)-NH2,
H-Asn-(Glu)5-des Pro36, Pro37, Pro38 [Met(O)14, Asp28] Exendin-4(1-39)-NH2,
des Pro36, Pro37, Pro38 [Met(O)14, Asp28] Exendin-4(1-39)-(Lys)6-NH2,
H-(Lys)6-des Pro36, Pro37, Pro38 [Met(O)14, Asp28] Exendin-4(1-39)-(Lys)6-NH2,
H-Asn-(Glu)5 des Pro36, Pro37, Pro38 [Met(O)14, Asp28] Exendin-4(1-39)-(Lys)6-NH2,
H-Lys6-des Pro36 [Met(O)14, Trp(O2)25, Asp28] Exendin-4(1-39)-Lys6-NH2,
H-des Asp28 Pro36, Pro37, Pro38 [Met(O)14, Trp(O2)25] Exendin-4(1-39)-NH2,
H-(Lys)6-des Pro36, Pro37, Pro38 [Met(O)14, Asp28] Exendin-4(1-39)-NH2,
H-Asn-(Glu)5-des Pro36, Pro37, Pro38 [Met(O)14, Trp(O2)25, Asp28] Exendin-4(1-39)-NH2,
des Pro36, Pro37, Pro38 [Met(O)14, Trp(O2)25, Asp28] Exendin-4(1-39)-(Lys)6-NH2,
H-(Lys)6-des Pro36, Pro37, Pro38 [Met(O)14, Trp(O2)25, Asp28] Exendin-4(S1-39)-(Lys)6-NH2,
H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1 C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

1-10. (canceled)

11. A method of closing the distal end of an ampoule comprising
providing an ampoule having a variable diameter head portion having a locating surface and an opening;
positioning a septum on the opening; and
securing the septum to the head portion with a ferrule using a plunger, where the plunger exerts a force in a proximal direction to press the ferrule on the head portion until the press causes the ferrule to contact the locating surface.

12. The method of claim 1 further characterized in that a rolling plate exerts a force in a distal direction to crimp the ferrule to the head portion.

13. An ampoule for containing a medicament comprising,
a constant diameter body;
a neck portion; and
a head portion located distally of the neck portion and having a variable, non-constant diameter.

14. The ampoule of claim 13 wherein the head portion has a first diameter (D1) and a second diameter (D2), wherein the first diameter (D1) is less than the second diameter (D2).

15. The ampoule of claim 14 wherein a diameter (D_{ND}) of the neck portion is less than the first and second diameters (D1, D2).

16. A system of medicament cartridges comprising
a first cartridge comprising a first ampoule according to claim 14, the first cartridge containing a first medicament; and
a second cartridge comprising a second ampoule according to claim 14, the second cartridge containing a second medicament,
wherein the second diameter (D2) of the first and second ampoules is the same dimension and the first diameter (D1) of the first and second ampoules is a different dimension.

17. The system of claim 16 wherein the first medicament has a first concentration and the second medicament has a second concentration, and where the second concentration is not equal to the first concentration.

18. The system of claim 15 wherein the first medicament in the first cartridge is different than the second medicament in the second cartridge.

19. The system of claim 16 wherein each of the first and second cartridges further comprises a septum mounted across a distal opening of the ampoule by a ferrule which conforms to the dimensions of the head portion.

20. The system of claim 19 wherein the ferrule includes the first and second diameters (D1, D2) of the respective ampoule.
